(19) United States
(12) Patent Application Publication
(10) Pub. No.: US 2010/0057564 A1
(43) Pub. Date: Mar. 4, 2010

(54) System and method for fitness motivation

(76) Inventor: Charles D. Godsey, JR., Greenwood, SC (US)

Correspondence Address: J. BENNETT MULLINAX, LLC, P. O. BOX 26029, GREENVILLE, SC, 29616-1029, US

(21) Appl. No.: 12/231,596

(22) Filed: Sep. 4, 2008

(51) Int. Cl. A61B 19/00; G06Q 30/00

(52) U.S. Cl. 705/14.53; 128/898

(57) ABSTRACT

A method of promoting the health of a user is provided. The method includes obtaining input from a user through the use of a website that is health related. Participation points are assigned for the user based upon the input obtained from the user. The participation points may be pooled with participation points of additional users. A participation point may be selected from the pool of participation points. The selected participation point is linked to a user such that a user is selected.

### FIELD OF THE INVENTION

The present invention relates generally to a system and method for motivating an individual in the realm of fitness. More particularly, the present application involves an on-line subscription based fitness community that functions to increase the health and wellness of its subscribers while consequently decreasing health care costs.

### BACKGROUND

Health care costs consume one seventh of our nation's gross domestic product. Aside from the economic aspect, health care is a critical element of the quality of life of every individual. Various economic and quality of life benefits result when an individual achieves and maintains an optimal level of fitness and health. As such, countless industries are devoted to improving the health and wellness of individuals.

On-line fitness communities are known for providing individuals access to information that can improve their well being. For example, dieting websites are known that allow users the ability to input the types and amounts of food they consume so that the amount of caloric intake in a one day period can be calculated. Websites of this type may provide the ability to determine the amount of calories present in a variety of different foods marketed under a variety of different brand names. Websites further provide the user with information relating to the amount of calories one may burn through the performance of various exercises. In this regard, the user can enter the type and amount of exercise undertaken and may be given a breakdown of the amount of calories burned. The levels of food intake and exercise can be quantified into numbers that can be tracked over a period of time to function as a tool in promoting weight loss and fitness. Further, the act of recording the amount of food consumed and exercise undertaken functions as a motivational tool to the user as it forces the user to examine their performance and encourages them to strive for a higher level of success based upon comparison with past results.

Dieting websites also offer other tools to members that function to promote weight loss and fitness. For example, body mass index calculators, ideal weight calculators, target heart rate calculators, and waist-to-hip ratio calculators can be employed in order to establish health related goals and provide information relating to a user's current level of health and fitness. Other tools can also be provided such as a weigh loss projection tool that allows a user to enter appropriate data to estimate their weight at future periods of time. Dieting websites also provide recipes, meal plans, diets, and articles related to health and fitness. Finally, some websites give users the ability to communicate with other individuals that are following the same fitness program so that their experiences and goals can be shared with one another. Although on-line access to fitness information is known, websites that promote the wellbeing of individuals lack sufficient tools for keeping an individual motivated throughout the course of their fitness program such that a permanent lifestyle change can be achieved. As such, there remains room for variation and improvement within the art.

### BRIEF DESCRIPTION OF THE DRAWINGS

A full and enabling disclosure of the present invention, including the best mode thereof, directed to one of ordinary skill in the art, is set forth more particularly in the remainder of the specification, which makes reference to the appended Figs. in which:

FIG. 1 is a schematic view of a system in accordance with one exemplary embodiment.

FIG. 2 is a flow chart that illustrates a method of improving the performance of a user in accordance with one exemplary embodiment.

FIG. 3 is a graphical user interface of a logging screen in accordance with one exemplary embodiment.

FIG. 4 is a graphical user interface of a household page in accordance with one exemplary embodiment.

FIG. 5 is a graphical user interface of a personal page in accordance with one exemplary embodiment.

Repeat use of reference characters in the present specification and drawings is intended to represent the same or analogous features or elements of the invention.

### DETAILED DESCRIPTION OF REPRESENTATIVE EMBODIMENTS

Reference will now be made in detail to embodiments of the invention, one or more examples of which are illustrated in the drawings. Each example is provided by way of explanation of the invention, and not meant as a limitation of the invention. For example, features illustrated or described as part of one embodiment can be used with another embodiment to yield still a third embodiment. It is intended that the present invention include these and other modifications and variations.

It is to be understood that the ranges mentioned herein include all ranges located within the prescribed range. As such, all ranges mentioned herein include all sub-ranges included in the mentioned ranges. For instance, a range from 100-200 also includes ranges from 110-150, 170-190, and 153-162. Further, all limits mentioned herein include all other limits included in the mentioned limits. For instance, a limit of up to 7 also includes a limit of up to 5, up to 3, and up to 4.5.

The present invention provides for a system 10 that can be used to improve the performance of a user 12. In one exemplary embodiment, the present system 10 can be used to improve the health of a user 12. The system 10 may be capable of obtaining input from the user 12 that is related to a health program to which the user 12 is taking part. The system 10 may assign participation points 38 to the various inputs of the user 12. The participation points 38 of various users taking part in the health program can be pooled and may be each counted as a single entry or vote. One of the participation points 38 may be randomly selected by the system 10 so that a random user 12 is selected. The selected user 12 may then be awarded a prize based upon the percentage of completion the user has attained on the health program to which they are taking part. In order to ensure the user 12 has indeed completed a certain percentage of the health program, verification may take place in the form of a visit to the user 12 and that is broadcast on a live television show. In this manner, the user 12 of the system 10 will be properly motivated in completing the health program through the award of a prize and through the knowledge that verification will be conducted.

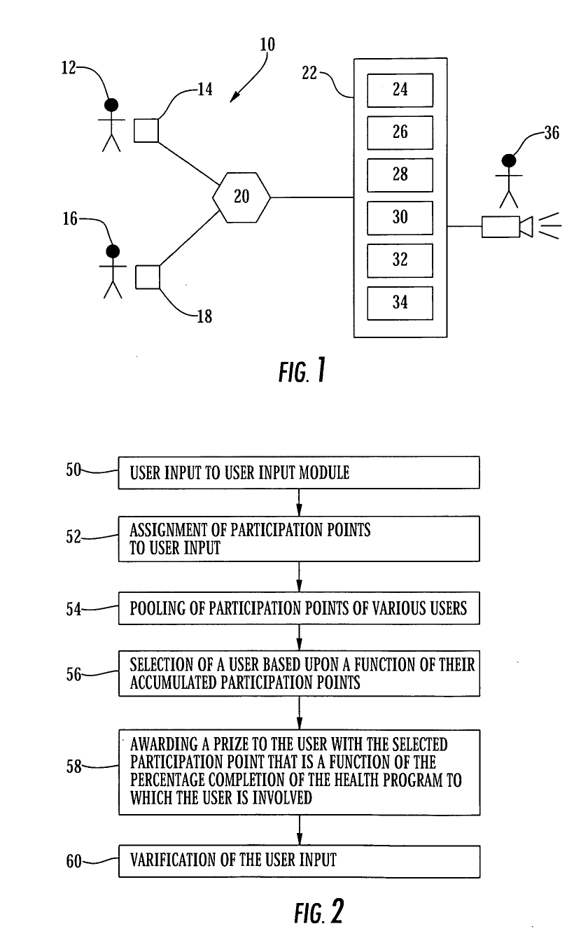

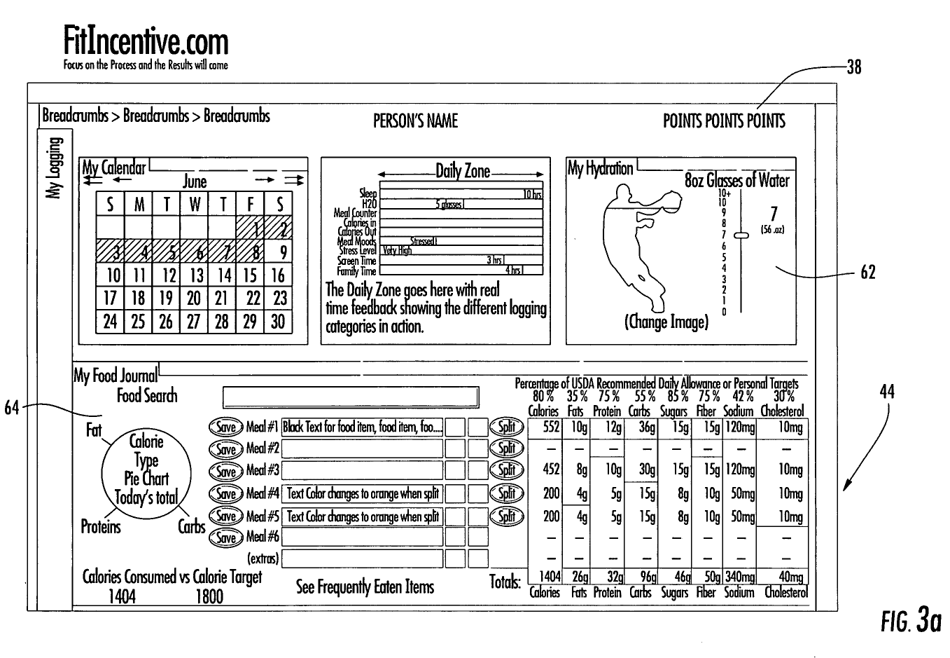

An exemplary embodiment of the system 10 is illustrated in FIG. 1. Here, a plurality of users 12, 16 can provide user input via user interfaces 14 and 16. The user interfaces 14 and 16 may be personal computers, cell phones, cable set top boxes or PDAs in accordance with various exemplary embodiments. Input from the user interfaces 14 and 16 may be communicated to a server 22 by way of a communication network 20. The communication network 20 may include portions of the Internet, a plain old telephone service (POTS) system, or a wireless network. The server 22 includes a plurality of modules 24, 26, 28, 30, 32 and 34 for use in carrying out various portions of the disclosed method. However, it is to be understood that the various modules may be included on any number of servers 22 and need not be all located on a single server 22 in accordance with other exemplary embodiments.

The user 12 may employ user interface 14 to access a website hosted by the server 22. The website provides access to an on-line health fitness community for purposes of promoting improved health of the user 12. Access to the website may be subscription based in accordance with certain exemplary embodiments. The website presents health related content to the user 12. For example, the website may provide access to weight management calculators, fitness expert content, progress charts, recipes, exercise instructions, message boards, and contact information for other users 16. The website also affords the user 12 the ability to log information therein. In this regard, the user 12 can make daily entries of health related content such as the entry of daily food and drink consumption, exercise conducted, sleep quantity and quality, tobacco and alcohol consumption, stress levels, caloric lifestyle changes, household time, and health program time. Additional information may be logged by the user 12. For example, the user 12 may provide body measurements or photographs to the website to assist in tracking progress.

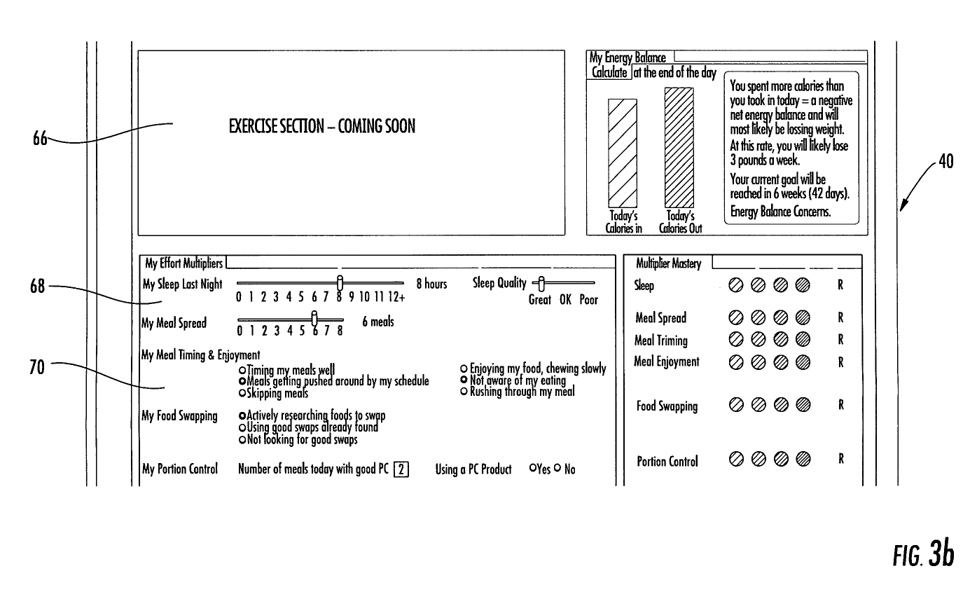

A method of improving the health of the user 12 is illustrated in FIG. 2. In step 50, the user 12 may input various information as previously discussed into a user input module 24 of a server 22. The system 10 may then proceed to step 52 in which a participation point assignment module 26 may function to assign participation points 38 to the user 12 input. For example, the participation points 38 may be assigned based upon profile creation, questionnaire completion, pictures posted, weekly goal assessment, daily logging, daily tip reading, successful article quiz completion, answer polls, user group creation, and/or posting in a user group. Participation points 38 may also be assigned if a user 12 adds a recipe, food item, or meal to the website. Further, if the user 12 contributes exercise instructions to the website participation points 38 may be given. Other participation point 38 assignments may be provided if the user 12 participates in a discussion on the website, has a website comment marked as helpful by other users 16, suggests an improvement to the program, or refers others to the program. Also, the user 12 may achieve the title of a “motivator” to another user 16 of the system 10. In this regard, the user 12 can provide assistance to user 16 in achieving their wellness goals though provision of encouragement or by acting as an educational resource. Participation points 38 may be provided if the user 12 becomes a motivator, obtains a motivator, or makes a motivational post on the website.

It is to be understood, however, that the assignment of participation points 38 to the aforementioned user 12 inputs are only exemplary and that others are possible in accordance with other exemplary embodiments. Various other user 12 inputs may be performed to which participation points 38 may be assigned. Further, the user 12 need not perform an affirmative action in order to earn a participation point 38. For example, participation points 38 may be assigned for each day the user 12 is a member of the health program, by chance based upon a random drawing, or through payment of a monthly bill for being a member of the interactive website. Also, the amount of participation points 38 may be varied in accordance with various exemplary embodiments. For example, the posting of a progress picture by a user 12 may be assigned twenty participation points 38 while the successful completion of a quiz related to fitness may only be assigned ten participation points 38. The assignment of participation points 38 may thus be made based on a variety of criteria and may be weighted in a desired manner.

The user 12 may accumulate participation points 38 over a certain time period. For example, the user 12 may accumulate participation points 38 over the course of a twelve week period. In accordance with other embodiments, the amount of participation points 38 may be totaled for time periods anywhere from twelve to sixteen weeks. In accordance with further exemplary embodiments, a timing cycle of up to six months may be used into which participation points 38 may be earned. Additional users 16 may also use the system 10 in a similar manner as user 12 to earn participation points 38 in a like regard. The participation points 38 from additional users 16 along with those from user 12 can be pooled in accordance with step 54 of the method. In this regard, a collection module 28 may function to keep track of the various participation points 38 from the users 12 and 16. Each of the participation points 38 may be linked to one of the users 12 and 16. Additionally or alternatively, the amount of participation points 38 each user 12 and 16 has may be totaled and can be assigned a percentage as the percentage of total participation points 38 owned by each of the users 12 and 16. Other methods of pooling and assigning owned participation points 38 are envisioned in accordance with other exemplary embodiments.

Once the participation points 38 are pooled or otherwise collected and assigned to each of the users 12 and 16, the method may proceed to step 56. Here, a selection module 30 may be employed by the system 10 in order to select at random one of the pooled participation points 38. As the selected participation point 38 may be linked to one of the users 12 or 16, the random selection of the pooled participation point 38 thus leads to the random selection of one of the users 12 or 16. Each one of the participation points 38 acts as a vote or entry such that a larger number of participation points 38 owned by the user 12 gives the user 12 a greater chance of being selected. In other arrangements, the users 12 and 16 may be assigned a certain percentage chance of winning based upon the percentage of participation points 38 they earned out the total accumulated amount of participation points 38. In other arrangements, one of the users 12 or 16 can be selected at random based upon a function of their accumulated participation points 38.

The selected user 12 may be awarded a prize by the system 10 in step 58. The amount of the prize can be determined based upon the completion percentage of the health program the user 12 performed during the period participation points 38 were earned. In this manner, the user 12 will be motivated to accumulate participation points 38 to win the awarded prize and thus will receive greater health benefits though greater use of the health program. A completion percentage module 32 can be used to calculate the user's 12 completion percentage. The completion percentage of the health program may be calculated in a variety of manners. For example, the health program can be broken down into various segments so that the amount of completion can be determined. If a user 14 satisfies enough elements in that particular segment, the segment may be deemed to have been completed. The amount of completed segments can be added up so that a completion percentage can be determined. Alternatively, the segments may be given a certain weight in establishing the completion percentage. In other arrangements, the elements themselves of each segment can be added up, possibly in a weighed fashion, to determine the completion percentage of the program. In certain embodiments, the percentage of completion of the segment can be determined based upon the percentage of completion of the elements in the segment and then, in turn, the weighted segments can be considered so that a completion percentage can be determined.

In accordance with one exemplary embodiment, one of the segments of the health program may be an educational component that includes elements such as reading articles on the website, watching videos on the website, and reading tips of the day. Another segment that may be considered may be a test scoring component that includes elements such as measured quiz scores the user 12 takes after reading various content on the website.

Other segments of the health program may be a social interactivity component that includes elements such as posting encouraging comments for other users 16, posting helpful forum questions and replies, acting as a motivator for other users 16, or interacting in various manners with other users 16. A further segment of the health program may be a logging component that includes elements such as the entry of the user's 12 meals, sleep amount and quality, hydration, and exercise. Another segment of the health program that may be incorporated into the percentage of completion may be an exercise component. The exercise component may include elements that are based on participation in activities such as regularly schedule walking, playing racquetball or performing aerobics. In other embodiments, the exercise component may include elements based upon how well the user 12 performs the exercise.

The completion percentage may thus be established based upon a function of the user input. The elements of the various segments can be assigned a level of importance so that certain elements count more towards the program completion percentage than other elements. The disclosed segments and elements of the program are only exemplary and others are possible. Although described with respect to certain segments and elements, it is to be understood that the completion percentage may be calculated in other manners in accordance with other exemplary embodiments. The prize awarded may be a function of the completion percentage. For example, if the prize is a cash prize, a maximum prize can be made possible. Should the user 12 score a completion percentage of 75% of the health program, the prize awarded the user 12 may be 75% of the maximum prize possible.

The method may include a verification step 60 in accordance with certain exemplary embodiments. Verification step 60 may be included in order to establish accuracy of the user input to ensure that the prize was properly awarded. In this regard, a television crew 36 may visit the user 12 to film the user 12 to ensure that the user input was accurate. The television crew 36 can film the user 12 on a live television show to ascertain whether the user 12 has improved his or her lifestyle through the health program in the manner that he or she indicated. In other embodiments, the television show need not be live. Also, the user 12 can be filmed and placed on the internet or on video without being placed onto a television show. As an example, the user 12 may be filmed while being weighted to ensure that their input weight was accurate. The user 12 may also be filmed while exercising to ensure that their user input was accurate. If the user 12 can only do five push-ups, when their user input indicated that they could do fifty, then the user input may be deemed to have been invalid. Other manners of deeming the user input invalid may be performed in accordance with other exemplary embodiments. If the user's input is deemed invalid, the user 12 will forfeit the entire amount of the prize. In other arrangements, a portion of the prize may be forfeit if the user input is invalid. The use of a live television crew 36 creates an element of accountability so that the user 12 will be properly motivated throughout the course of the health program. The user 12 may thus be required to perform some type of exercise to for their win to be properly validated. Although described as being validated through the use of a live television program, it is to be understood that other forms of validation are possible in accordance with other exemplary embodiments. The method thus motivates the user 12 to work at the health program by providing the opportunity for a prize based upon the user's level of involvement with the health program and also ensures the user 12 is deterred from fabricating user input through the employment of a validation step.

Once a prize has been awarded, the number of participation points 38 may be reset to zero for each user 12 and 16 and a new amount of time may be started into which participation points 38 may be earned. As such, twelve or sixteen week cycles may be tracked during which participation points 38 are accumulated and a prize awarded. Although described as employing a twelve or sixteen week cycle, it is to be understood that cycles of varying length may be used in other embodiments. It is to be understood that the prize awarded the winning user 12 or 16 may be a single prize or may be multiple prizes. Further, a single winning user 12 or 16 need not be selected in accordance with other exemplary embodiments. For example, multiple prizes may be awarded to multiple winning users 12 or 16 which are based on the users 12 and 16 completion percentages.

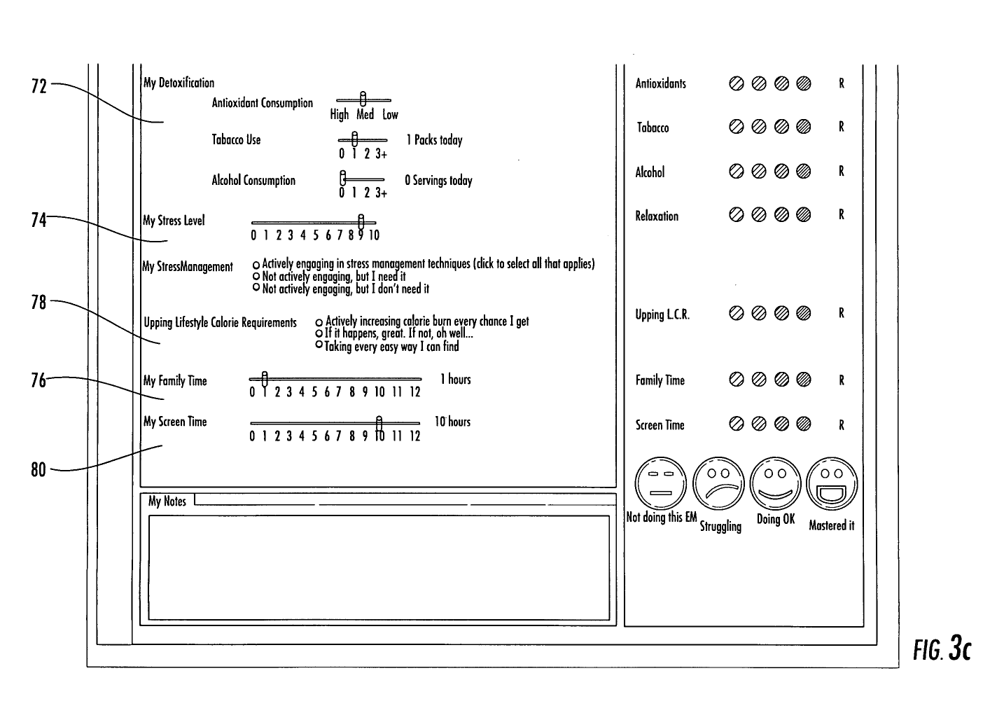

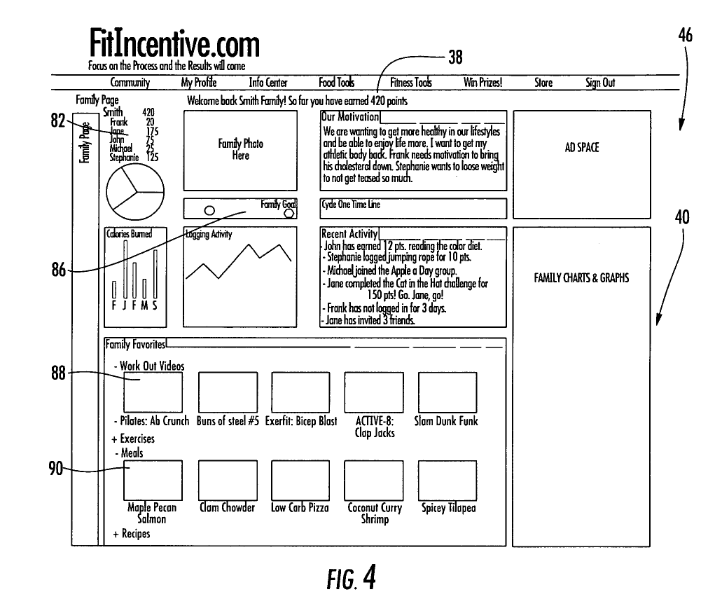

The user 12 may employ a device with a user interface 14 that presents the user 12 with a graphical user interface 40. The graphical user interface 40 may be presented through instructions by the graphical interface display module 34 of the server 22. The graphical user interface 40 can display a logging page 44 as shown in FIG. 3 to allow the user 12 to log information into the website. The logging page 44 can present various inputs to the user 12 to allow the user 12 to enter user input health related information into the website. For example, the logging page 44 can have a hydration input 62 that allows the user 12 to enter the number of glasses of water consumed that day or over any time period. A food input 64 can also be presented to receive user input related to the type, amount and brand of food consumed. The food input 64 may also present a graphical display that breaks down the daily allowances consumed as per information received by the food input 64. The logging page 44 may also include an exercise input 66 that obtains information on the types and amounts of exercise performed by the user 12. A comparison of the exercise and food intake may be made so that the user 12 can be presented with feedback regarding their energy balance and weight loss or gain as per the input information.

The logging page 44 includes other sections designed for receiving user input on other health related matters. A sleep input 68 is present into which the user 12 may input the hours of sleep obtained the night before along with the quality of the sleep obtained. A meal input 70 can be presented to the user 12 to determine the meal spread, timing, enjoyment, and portions consumed. Also present is a detox input 72 that receives input from the user 12 regarding tobacco consumption, alcohol consumption, and antioxidant consumption. Further, a stress input 74 may be present to receive the user's 12 stress level and to request information on whether the user 12 is performing stress management techniques. A family time input 76 can receive information relating to the amount of family time spent that day by the user 12. Also included is a lifestyle change input 78 that receives information from the user 12 to determine whether they have made any calorie burning adjustments in their lifestyle such as making a conscious choice to take the stairs on a daily basis instead of taking the elevator. Further, a screen input 80 can be present in order to receive information relating to the amount of time spent by the user 12 on the website. This information may be tracked automatically in other embodiments. The various inputs may be received through entry into a text box, selection of a number on a sliding scale, selection of an entry through a drop down box, or though any other mechanism. It is to be understood, however, that the disclosed inputs on the logging page 44 are only exemplary and that other inputs may be present in other exemplary embodiments.

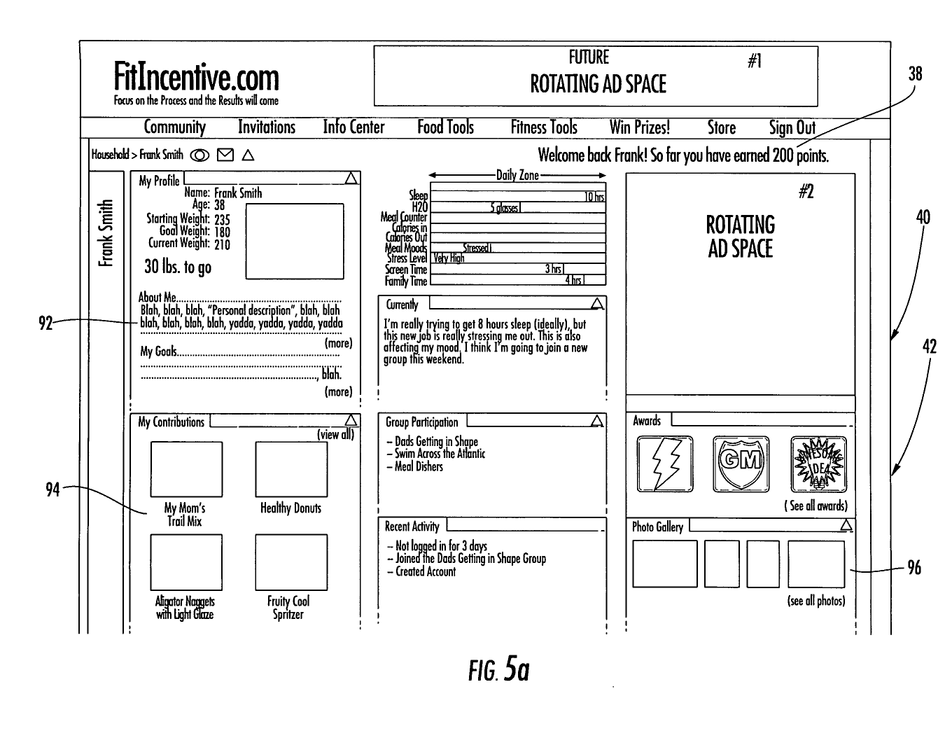

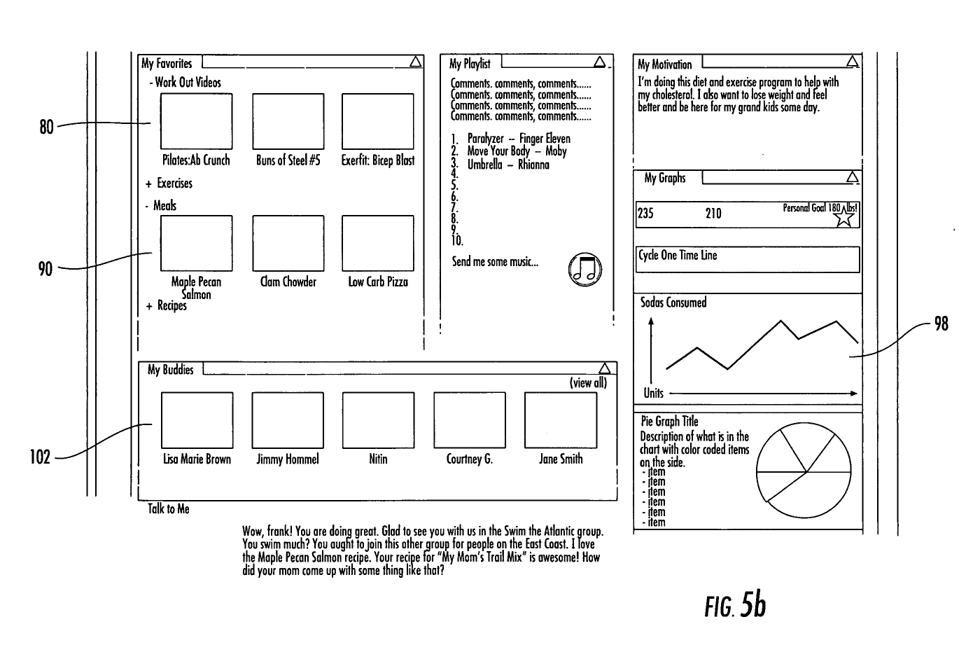

The user 12 may be an individual user 12 or may be a member of a household that has a household account. A household page 46 is illustrated in FIG. 4. The household page 46 can provide information relative to the household's participation in the health program. For example, the amount of participation points 38 can be viewed on the household page 46. The participation points 38 may be the total amount of participation points 38 accumulated by all of the household members added together. A participation point breakdown 82 can be displayed on the household page 46 to illustrate the amount of participation points 38 earned by each member of the household. The system 10 and method can be arranged so that the participation points 38 used to determine the selected user 12 can be attributed to the household as a unit, or may be attributable to each individual member of the household so that instead of selecting a winner that is a household for the prize, an individual member of the household may be selected as the selected user 12. The household page 46 may also have a calorie breakdown 84 that shows the amount of calories consumed or burned for each household member, and can have a household goal progression 86 that tracks the progress of the household's performance in the health program with respect to their goal. Additional information may be displayed on the household page 46. Video content including exercise videos 88 can be located on the household page 46 for access thereto. Also, recipes 90 may be presented and can be accessed on the household page 46. It is to be understood that the information displayed on the household page 46 is only exemplary and that other types of information are possible in accordance with other exemplary embodiments.

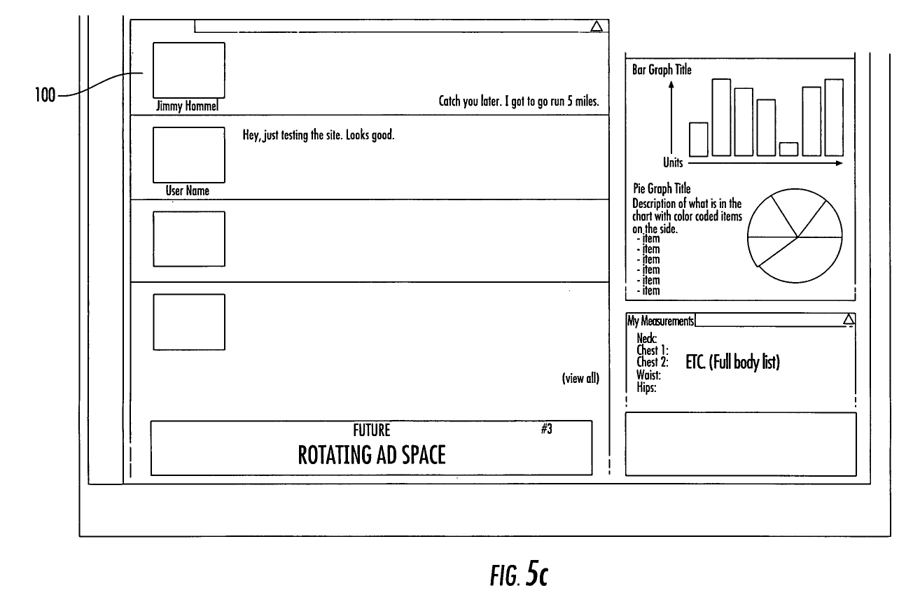

FIG. 5 is a personal page 42 displayed by the user interface 14 of a single individual user 12. The user's 12 personal page 42 can be set to private so that only the user 12 can view the personal page 42. Alternatively, the user 12 can set his or her personal page 42 to public and may instruct that only certain information is displayed. The amount of participation points 38 earned by the user 12 can be displayed. The personal page 42 may include weight goals 92 of the user 12 that display the user's starting weight, current weight, and goal weight. The amount of weight to lose and the projecting time to arrive at the goal may be shown. Also shown are contributions 94 made by the user 12 to the website. For example, the user 12 may have contributed a recipe or work out video to the website to earn participation points 38. Also Photographs 96 of the user 12 can be displayed on his or her personal page 42. Other information such as the amount of soft drinks consumption 98 can be tracked by the user 12 and displayed on the personal page 42. The personal page 42 also displays messages 100 received by the user 12 from other users 16 of the health program or from an administrator of the system 10. The user 12 may have various friends 102 that are users 16 of the system 10 to which the user 12 may communicate. It is to be understood that the information displayed on the personal page 42 is only exemplary and that other information may be displayed in accordance with other exemplary embodiments.

The health program may seek to improve the fitness, nutrition, exercise, wellness, relational well being, lifestyle management, business success, financial standing, athletic performance and workplace performance of the user 12. Although described as incorporating a health program, the system 10 may be modified in other exemplary embodiments so that a program other than a health program is implemented. For example, the system 10 can be modified so that the program the user 12 is taking part in is a workplace performance program. In this regard, if the user 12 is a teacher the participation points 38 can be assigned based upon the amount of planning time the user 12 spends, educational presentations given to other teachers, time spent at school activities, student progress, and the like. The system 10 will function in a similar manner so that a user 12 will be selected and awarded a prize based upon program completion so that the user 12 will be properly motivated though the course of the program. As such, it is to be understood that other exemplary embodiments are possible in which the program is not health related. Further, the monitoring, prize awarding and verification may be done by a third party, or the users 12 and 16 in accordance with various embodiments.

When implemented by a company, the system 10 can be used to evaluate employee compliance and participation in a health program or other program run by system 10. The company may view the information contained within system 10 of their employees so long as employee privacy as protected under the law is maintained.

While the present invention has been described in connection with certain preferred embodiments, it is to be understood that the subject matter encompassed by way of the present invention is not to be limited to those specific embodiments. On the contrary, it is intended for the subject matter of the invention to include all alternatives, modifications and equivalents as can be included within the spirit and scope of the following claims.

What is claimed:

1. A method of promoting the health of a user, comprising:
obtaining input from a user through the use of a website, wherein the website is health related;
assigning participation points for the user based upon the input obtained from the user;
pooling the participation points of the user with participation points of additional users; and
selecting a participation point from the pool of participation points, wherein the selected participation point is linked to a user such that a user is selected.

2. The method as set forth in claim 1, further comprising:
awarding a prize to the selected user, wherein the amount of the prize is based upon the user's completion percentage of a health program.

3. The method as set forth in claim 2, wherein the health program has an educational component, a test scoring component, an exercise component, a website social interactivity component, and a website logging component, and wherein the user's completion percentage of the health program is calculated based upon the amount of the educational component, the amount of the test scoring component, the amount of the exercise component, the amount of the website social interactivity component, and the amount of the website logging component performed by the user.

4. The method as set forth in claim 2, further comprising:
visiting the user to verify the user's input; and
forfeiting the prize awarded to the user if the user's input is not verified.

5. The method as set forth in claim 4, wherein visiting the user includes filming the user for broadcast.

6. The method as set forth in claim 1, wherein the input from the user to which participation points are assigned includes information relating to exercise performed by the user.

7. The method as set forth in claim 1, wherein the input from the user to which participation points are assigned includes information relating to a profile created by the user, progress pictures posted by the user, daily logging by the user, sending messages to other users, adding a recipe, and reading daily tips.

8. A method of motivating a user, comprising:
obtaining input from users through the use of a website, wherein the website is directed towards a program to which the users are involved;
assigning participation points for the users based upon the input obtained from the users;
selecting one of the users based upon a function of the number of participation points accumulated; and
awarding a prize to the selected user, wherein the amount of the prize is based upon the user's completion percentage of the program.

9. The method as set forth in claim 8, wherein the program is a health related program.

10. The method as set forth in claim 8, further comprising:
visiting the user to verify the user's input; and
forfeiting the prize awarded to the user if the user's input is not verified.

11. The method as set forth in claim 10, wherein visiting the user includes filming the user for broadcast.

12. The method as set forth in claim 8, wherein the input from the users to which participation points are assigned includes information relating to exercise performed by the users.

13. The method as set forth in claim 8, wherein selecting a participation point includes pooling all of the assigned participation points of the users and randomly selecting one of the assigned participation points such that each assigned participation point has an equal chance of being randomly selected.

14. The method as set forth in claim 8, wherein the input from the users to which participation points are assigned includes information relating to a profile created by the users, progress pictures posted by the users, daily logging by the users, sending messages, adding a recipe, and reading daily tips.

15. A system for promoting the health of a user, comprising:
a user input module configured for receiving input from a user from a health related website;
a participation point assignment module configured for assigning participation points to input received by the user input module;
a collection module configured for pooling a plurality of participation points of various users; and
a selection module configured for randomly selecting a participation point from the plurality of participation points pooled by the collection module, wherein the selected participation point represents a selected user.

16. The system as set forth in claim 15, further comprising a completion percentage module configured for determining the selected user's percentage completion of a health program, wherein the completion percentage module is configured for determining an amount of a prize award for the selected user based upon the selected user's percentage completion.

17. The system as set forth in claim 16, wherein the health program has an educational component, a test scoring component, an exercise component, a website social interactivity component, and a website logging component, and wherein the user's completion percentage of the health program is calculated based upon the amount of the educational component, the amount of the test scoring component, the amount of the exercise component, the amount of the website social interactivity component, and the amount of the website logging component performed by the user.

18. The system as set forth in claim 15, further comprising:
a plurality of user interfaces configured to allow the users to send user input to the user input module; and
a communication network that enables communication between the plurality of user interfaces and the user input module;
wherein the user input module, the participation point assignment module, the collection module, and the selection module are located on a server.

19. The system as set forth in claim 15, wherein the input from the user to which participation points are assigned includes information relating to exercise performed by the user.

20. The system as set forth in claim 15, wherein the input from the user to which participation points are assigned includes information relating to a profile created by the user, progress pictures posted by the user, daily logging by the user, sending messages to other users, adding a recipe, and reading daily tips.
